(19) United States
(12) Patent Application Publication
(10) Pub. No.: US 2023/0395220 A1
(43) Pub. Date: Dec. 7, 2023

(54) SYSTEMS AND METHODS FOR PROVIDING AN INTERACTIVE DIGITAL PERSONALIZED EXPERIENCE INTERFACE FOR USERS TO ENGAGE IN VARIOUS ASPECTS OF BEHAVIORAL HEALTHCARE

(71) Applicant: Evernorth Strategic Development, Inc., St. Louis, MO (US)

(72) Inventor: Christina M. Vallery, Eastsound, WA (US)

(21) Appl. No.: 18/205,388

(22) Filed: Jun. 2, 2023

Related U.S. Application Data
(60) Provisional application No. 63/348,365, filed on Jun. 2, 2022.

(51) Int. Cl. G16H 20/00; G16H 10/20; G16H 40/67; G06T 13/40

(52) U.S. Cl. CPC G16H 20/00; G16H 10/20; G16H 40/67; G06T 13/40

(57) ABSTRACT

A method includes receiving a prescription notification indicating information corresponding to the at least one prescription and information corresponding to the user. The method also includes identifying, based on the prescription notification, at least one health condition of the user. The method also includes, in response to the at least one health condition corresponding to at least one health condition of a plurality of predetermined health conditions, generating a message including instructions for downloading a user application. The method also includes, in response to an indication that the user initiated the user application, providing, at an application setup interface, a plurality of data gathering queries. The method also includes generating, using the user responses, a data structure corresponding to the user. The method also includes providing, based on the data structure corresponding to the user, a personalized experience interface.

### CROSS-REFERENCE TO RELATED APPLICATION

The present disclosure claims priority to U.S. Provisional Patent Application Ser. No. 63/348,365, filed Jun. 2, 2022, which is incorporated herein by reference in its entirety.

### TECHNICAL FIELD

This disclosure relates to interactive digital personalized experience interfaces, and in particular for providing an interactive digital personalized experience interface for users to engage in various aspects of behavioral healthcare.

### BACKGROUND

Healthcare management is increasingly becoming a complex and important aspect of daily life. As health aspects of conditions of an individual change over time, the individual typically engages in various healthcare treatments, procedures, therapies, and the like. Managing such aspects of the health of the individual can be burdensome, complex, and/or difficult and may result in noncompliance with healthcare treatments, procedures, therapies, and the like, which may provide less than optimal healthcare for the individual.

### SUMMARY

This disclosure relates generally to interactive digital personalized experience interfaces.

An aspect of the disclosed embodiments includes a method for providing an interactive digital personalized experience interface. The method includes receiving, responsive to a user filling at least one prescription, a prescription notification indicating information corresponding to the at least one prescription and information corresponding to the user. The method also includes identifying, based on the prescription notification, at least one health condition of the user. The method also includes, in response to the at least one health condition corresponding to at least one health condition of a plurality of predetermined health conditions, generating a message including instructions for downloading a user application. The method also includes communicating, to a user account associated with the user, the message and, in response to an indication that the user initiated the user application, providing, at an application setup interface, a plurality of data gathering queries. The method also includes storing user responses to the data gathering queries and generating, using the user responses, a data structure corresponding to the user. The method also includes generating, based on the data structure corresponding to the user, a personalized experience interface, and providing, at a display of a computing device associated with the user, the personalized experience interface.

Another aspect of the disclosed embodiments includes a system for providing an interactive digital personalized experience interface. The system includes a processor, and a memory. The memory includes instructions that, when executed by the processor, cause the processor to: receive, responsive to a user filling at least one prescription, a prescription notification indicating information corresponding to the at least one prescription and information corresponding to the user; identify, based on the prescription notification, at least one health condition of the user; in response to the at least one health condition corresponding to at least one health condition of a plurality of predetermined health conditions, generate a message including instructions for downloading a user application; communicate, to a user account associated with the user, the message; in response to an indication that the user initiated the user application, provide, at an application setup interface, a plurality of data gathering queries; store user responses to the data gathering queries; generate, using the user responses, a data structure corresponding to the user; generate, based on the data structure corresponding to the user, a personalized experience interface; and provide, at a display of a computing device associated with the user, the personalized experience interface.

Another aspect of the disclosed embodiments includes an apparatus for providing an interactive digital personalized experience interface. The apparatus includes one or more processors, and a memory. The memory includes instructions that, when executed by the one or more processors, cause the one or more processors to, respectively or collectively: receive a prescription notification indicating information corresponding to at least one prescription and information corresponding to a user associated with the at least one prescription; identify, based on the prescription notification, at least one health condition of the user; in response to the at least one health condition corresponding to at least one health condition of a plurality of predetermined health conditions, generate a message including instructions for downloading a user application; communicate, to a user account associated with the user, the message; in response to an indication that the user initiated the user application, provide, at an application setup interface, a plurality of data gathering queries; generate, using the user responses, a data structure corresponding to the user; generate, based on the data structure corresponding to the user, a personalized experience interface; and provide, at a display of a computing device associated with the user, the personalized experience interface.

These and other aspects of the present disclosure are disclosed in the following detailed description of the embodiments, the appended claims, and the accompanying figures.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1A generally illustrates a functional block diagram of a system including a high-volume pharmacy according to the principles of the present disclosure.

FIG. 1B generally illustrates a computing device according to the principles of the present disclosure.

FIG. 2 generally illustrates a functional block diagram of a pharmacy fulfillment device, which may be deployed within the system of FIG. 1A.

FIG. 3 generally illustrates a functional block diagram of an order processing device, which may be deployed within the system of FIG. 1A.

FIG. 4 generally illustrates a personalized healthcare experience method according to the principles of the present disclosure.

FIG. 5 generally illustrates a personalized experience interface according to the principles of the present disclosure.

FIGS. 6A and 6B are graphical representations of example recurrent neural networks for generating personalized healthcare.

FIG. 7 is a graphical representation of layers of an example long short-term memory (LSTM) machine learning model.

FIG. 8 is a flowchart illustrating an example process for training a machine learning model.

FIG. 9 is a block diagram of an example personalized experience interface selection system that may be deployed within the systems described herein, according to some examples.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

As described, healthcare management is increasingly becoming a complex and important aspect of daily life. As health aspects of conditions of an individual change over time, the individual typically engages in various healthcare treatments, procedures, therapies, and the like. Managing such aspects of the health of the individual can be burdensome, complex, and/or difficult and may result in noncompliance with healthcare treatments, procedures, therapies, and the like, which may provide less than optimal healthcare for the individual.

Accordingly, systems and methods, such as those described herein, configured to provide a personalized experience interface for managing aspects of the healthcare of an individual, may be desirable. In some embodiments, the systems and methods described herein may be configured to provide proprietary personalized care including one or more personalized experience components generated and/or selected based on enrollment criteria, insurance plan structure, care pathway considerations, additional care pathway considerations, and/or preferences stated or provided by a user. The systems and methods described herein may be configured to provide dynamic engagement components as a part of a checklist or wellness dashboard.

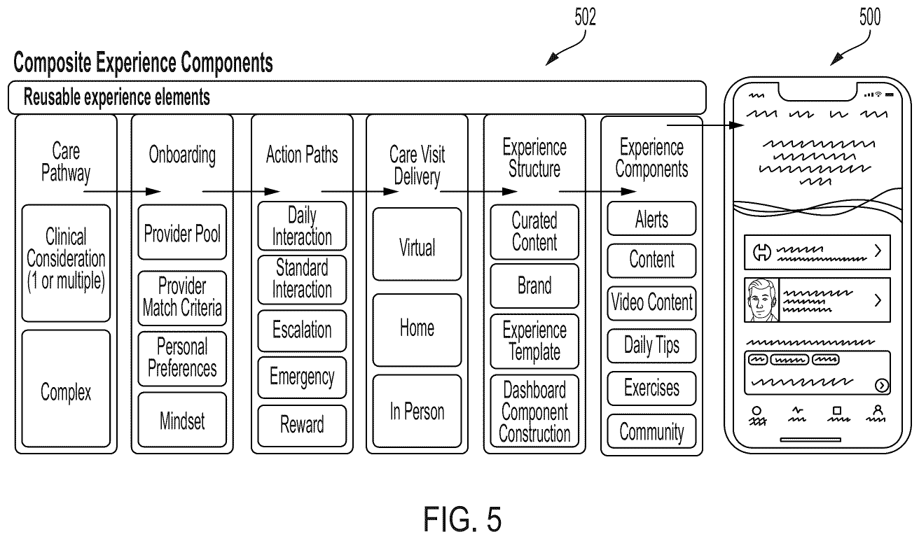

As is generally illustrated in FIG. 5, the systems and methods described herein may be configured to provide composed experience and composite customer experience, generally illustrated at 502. For example, the systems and methods described herein may be configured to identify one or more care pathways (e.g., based on one or more health conditions of the user). In response to identifying the one or more care pathways, the systems and methods described herein may be configured to onboard the user, which may include, based on input received from the user, identifying a provider pool, matching the user with a provider, receiving personal preferences of the user, and determining a mindset of the user.

The systems and methods described herein may be configured to identifying one or more action paths that may include one or more daily interactions (e.g., including one or more queries provided to the user via a personalized experience interface 500), one or more standard interactions, one or more escalation actions (e.g., based on responses from the user to the daily interactions and/or the standard interactions), one or more emergency actions (e.g., based on responses from the user to the daily interactions and/or the standard interactions), and/or one or more reward actions.

The systems and methods described herein may be configured to provide one or more care visit delivery settings including one or more virtual care visit settings, one or more in home care visit settings, and one or more in-person care visit settings.

The systems and methods described herein may be configured to identify an experience structure that may include curated content, brand content, an experience template, and/or dashboard component construction. The systems and methods described herein may be configured to identifying one or more experience components that may include one or more alerts, content, video content, daily tips, exercises, community components, and/or any other suitable components.

The systems and methods described herein may be configured to generate the personalized experience interface 500 based on the composite experience components 502. The personalized experience interface 500 may be configured to provide a personalized experience for engaging the user in actively participating in the treatment, maintenance, therapy, and other aspects of the health of the user.

In some embodiments, the systems and methods described herein may be configured to provide experience structure and experience components. In some embodiments, the systems and methods described herein may be configured to provide identity content based on a user eligibility (e.g., based on a primary care pathway, a secondary care pathway, prescription claim data, electronic medical record data, and/or the like), demographic health persona construct associated with the user (e.g., including general demographics (e.g., age, family history, diagnoses, body mass index, lifestyle factors, social determinants of health, medication, and/or the like) and/or data-driven health dimensions (e.g., general wellbeing, access to technology, stress associated with benefits use, social support, financial comfort and income, trust in healthcare, and/or the like), mindset persona construct of the user (e.g., including persistent denial, angry, fearful, overwhelmed, lonely, independent, inquisitive, determined, and/or the like), self-reported data (e.g., during onboarding) of the user (e.g., including username, electronic mail address, password, social security number, plan or member identification number, one or more models (e.g., standard clinical, relationship focus path, behavioral change focus path, aspirational goals focus path, and/or the like) and/or the like), and/or the like.

In some embodiments, the systems and methods described herein may be configured to receive, responsive to the user filling at least one prescription, a prescription notification indicating information corresponding to the at least one prescription and information corresponding to the user. The information corresponding to the at least one prescription may include at least prescription identification information, dosing information, other suitable information, and the like. The information corresponding to the user may include at least user statistical information, user contact information, other suitable information, and the like.

The systems and methods described herein may be configured to identify, based on the prescription notification, at least one health condition of the user. The systems and methods described herein may be configured to, in response to the at least one health condition corresponding to at least one health condition of a plurality of predetermined health conditions, generate a message including instructions for downloading a user application. The plurality of predetermined health conditions may include at least one of at least one behavioral health condition, at least one musculoskeletal health condition, and/or at least one other health condition. The at least one behavioral health condition may correspond to at least one of depression, generalized anxiety, bipolar disorder, at least one other behavioral health condition, and/or any other suitable behavioral health condition. The at least one musculoskeletal health condition may correspond to at least one of back pain, neck pain, joint pain, at least one other musculoskeletal health condition, and/or any other suitable musculoskeletal health condition. The at least one other health condition corresponds to at least one of cancer, diabetes, heart failure, heart disease, chronic obstructive pulmonary disease, covid 19, at least one other health condition, and/or any other suitable health condition.

The systems and methods described herein may be configured to communicate, to a user account associated with the user, the message. The user account associated with the user may include an electronic mail account, a pharmacy account, a health insurance account, at least one other user account, and/or the like. The systems and methods described herein may be configured to, in response to an indication that the user initiated the user application, provide, at an application setup interface, a plurality of data gathering queries. The data gathering queries may include at least one questions corresponding to a mental or emotional state of the user, a pain level of the user, an energy level of the user, and/or the like. Additionally, or alternatively, the data gathering queries may include one or more questions corresponding to the age of the user, the family history of the user, the location of the user, and/or the like. Additionally, or alternatively, the data gathering queries may correspond to a level of financial comfort of the user, a level of comfort of the user with insurance benefits, demographics of the user, technology access of the user, and/or the like. It should be understood that the data gathering queries may correspond to any suitable information to be gathered from the user.

The systems and methods described herein may be configured to store, in a database or other suitable data store, user responses to the data gathering queries. The systems and methods described herein may be configured to generate, using the user responses, a data structure corresponding to the user. The systems and methods described herein may be configured to generate, based on the data structure corresponding to the user, a personalized experience interface. In some embodiments, the personalized experience interface may comprise a portable health record comprising the health record of the user. The personalized experience interface may include one or more personalized components that include at least a daily interaction component, a relevant resource component, a dynamic selection component, and/or the like. The dynamic selection component may include at least one general selection and/or, responsive to the at least one health condition, at least one health condition specific component.

The systems and methods described herein may be configured to provide, at a display of a computing device associated with the user, the personalized experience interface. The personalized experience interface may correspond to a user avatar that models a biological identity of the user. The user avatar may correspond to personalized, intelligence associated with a predictive healthcare driven model and may be based on a data driven, dynamic patient survey. The systems and methods described herein may be configured to provide, at a metaverse (e.g., a three-dimensional virtual environment), access to at least one three-dimensional environment feature using the user avatar. The at least one three-dimensional environment feature may include a virtual healthcare provider office, a virtual healthcare provider, at least one other three-dimensional environment feature, and/or the like. The virtual healthcare provider may include an avatar corresponding to a healthcare provider (e.g., in the real-world), an avatar corresponding to an artificially intelligent healthcare provider (e.g., a virtual healthcare provider driving my artificial intelligence), and/or the like.

The systems and methods described herein may be configured to identify, using a first set of user responses, a healthcare provider pool. The systems and methods described herein may be configured to adjust at least some of the data gathering queries based on the first set of user responses. The systems and methods described herein may be configured to identify, responsive to a second set of user responses corresponding to at least some of the adjusted data gathering queries, a subset of healthcare providers of the healthcare provider pool that match with the user.

In some embodiments, the personalized experience interface includes, at least, information corresponding to each of the subset of healthcare providers. The systems and methods described herein may be configured to schedule, responsive to a selection of at least one healthcare provider of the subset of healthcare providers by the user using the personalized experience interface, at least one appointment, for the user, with the selected at least one healthcare provider.

The systems and methods described herein may be configured to receive feedback from the user corresponding to at least one of a component of the personalized experience interface and an appointment with the selected at least one healthcare provider. The systems and methods described herein may be configured to refine, responsive to the feedback, the data gathering queries, the personalized experience interface, and/or any other suitable feature. The systems and methods described herein may be configured to identify, based on the feedback, at least one other healthcare provider from the healthcare provider pool.

The systems and methods described herein may be configured to select, using the data structure corresponding to the user, at least one suggestive aspect of the personalized experience interface. The systems and methods described herein may be configured to provide, at the personalized experience interface, the at least one suggestive aspect. The at least one suggestive aspect may include at least one of a color, a dynamic background, at least one other suggestive aspect, and/or the like. Additionally, or alternatively, the at least one suggestive aspect may be configured to provide, responsive to the user engaging with the at least one suggestive aspect, at least one of a physical response of the user and a mental response of the user.

The systems and methods described herein may be configured to generate or refine one or more of at least one aspect of the data gathering queries, at least one aspect of the personalized experience interface, and/or the like, using at least one machine learning model configured to use at least some of the user responses and at least some responses provided by other users to dynamically generate or refine the at least one of at least one aspect of the data gathering queries and at least one aspect of the personalized experience interface.

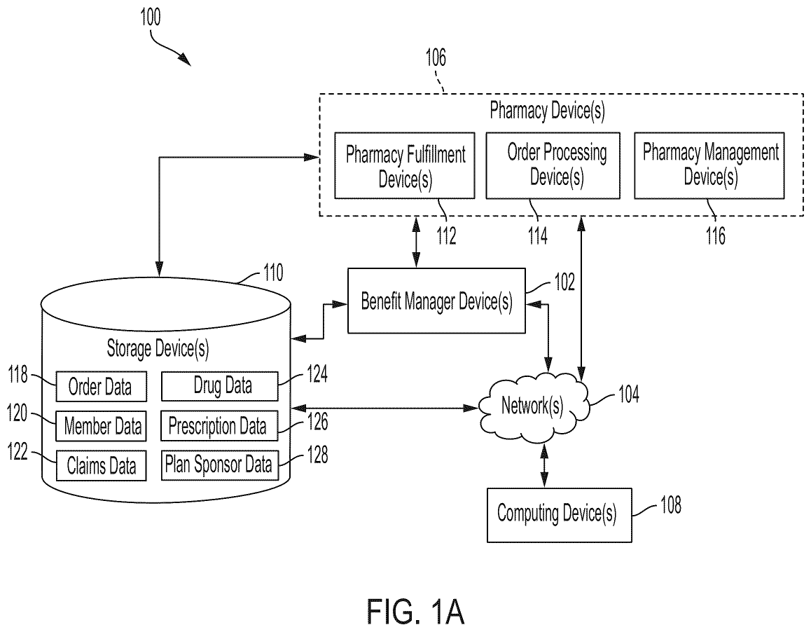

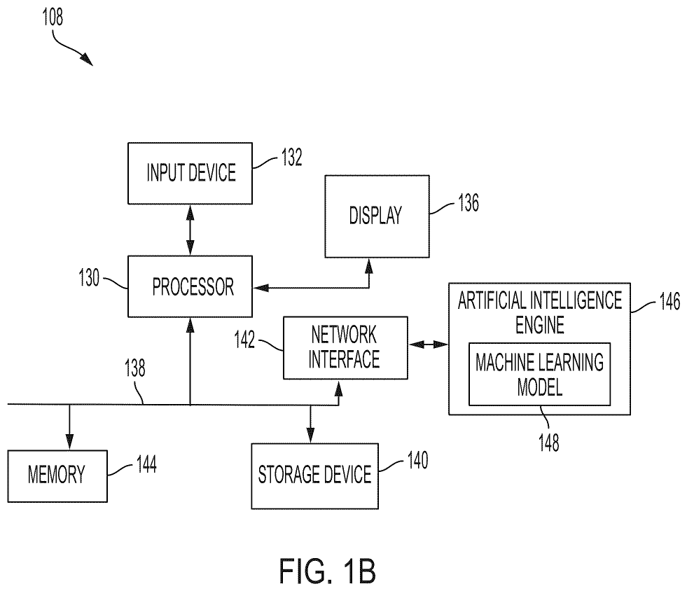

FIG. 1A is a block diagram of an example implementation of a system 100 for a high-volume pharmacy. While the system 100 is generally described as being deployed in a high-volume pharmacy or a fulfillment center (for example, a mail order pharmacy, a direct delivery pharmacy, etc.), the system 100 and/or components of the system 100 may otherwise be deployed (for example, in a lower-volume pharmacy, etc.). A high-volume pharmacy may be a pharmacy that is capable of filling at least some prescriptions mechanically. The system 100 may include a benefit manager device 102 and a pharmacy device 106 in communication with each other directly and/or over a network 104. The system 100 may also include a storage device 110.

The benefit manager device 102 is a device operated by an entity that is at least partially responsible for creation and/or management of the pharmacy or drug benefit. While the entity operating the benefit manager device 102 is typically a pharmacy benefit manager (PBM), other entities may operate the benefit manager device 102 on behalf of themselves or other entities (such as PBMs). For example, the benefit manager device 102 may be operated by a health plan, a retail pharmacy chain, a drug wholesaler, a data analytics or other type of software-related company, etc. In some embodiments, a PBM that provides the pharmacy benefit may provide one or more additional benefits including a medical or health benefit, a dental benefit, a vision benefit, a wellness benefit, a radiology benefit, a pet care benefit, an insurance benefit, a long term care benefit, a nursing home benefit, etc. The PBM may, in addition to its PBM operations, operate one or more pharmacies. The pharmacies may be retail pharmacies, mail order pharmacies, etc.

Some of the operations of the PBM that operates the benefit manager device 102 may include the following activities and processes. A member (or a person on behalf of the member) of a pharmacy benefit plan may obtain a prescription drug at a retail pharmacy location (e.g., a location of a physical store) from a pharmacist or a pharmacist technician. The member may also obtain the prescription drug through mail order drug delivery from a mail order pharmacy location, such as the system 100. In some embodiments, the member may obtain the prescription drug directly or indirectly through the use of a machine, such as a kiosk, a vending unit, a mobile electronic device, or a different type of mechanical device, electrical device, electronic communication device, and/or computing device. Such a machine may be filled with the prescription drug in prescription packaging, which may include multiple prescription components, by the system 100. The pharmacy benefit plan is administered by or through the benefit manager device 102.

The member may have a copayment for the prescription drug that reflects an amount of money that the member is responsible to pay the pharmacy for the prescription drug. The money paid by the member to the pharmacy may come from, as examples, personal funds of the member, a health savings account (HSA) of the member or the member's family, a health reimbursement arrangement (HRA) of the member or the member's family, or a flexible spending account (FSA) of the member or the member's family. In some instances, an employer of the member may directly or indirectly fund or reimburse the member for the copayments.

The amount of the copayment required by the member may vary across different pharmacy benefit plans having different plan sponsors or clients and/or for different prescription drugs. The member's copayment may be a flat copayment (in one example, $10), coinsurance (in one example, 10%), and/or a deductible (for example, responsibility for the first $500 of annual prescription drug expense, etc.) for certain prescription drugs, certain types and/or classes of prescription drugs, and/or all prescription drugs. The copayment may be stored in the storage device 110 or determined by the benefit manager device 102.

In some instances, the member may not pay the copayment or may only pay a portion of the copayment for the prescription drug. For example, if a usual and customary cost for a generic version of a prescription drug is $4, and the member's flat copayment is $20 for the prescription drug, the member may only need to pay $4 to receive the prescription drug. In another example involving a worker's compensation claim, no copayment may be due by the member for the prescription drug.

In addition, copayments may also vary based on different delivery channels for the prescription drug. For example, the copayment for receiving the prescription drug from a mail order pharmacy location may be less than the copayment for receiving the prescription drug from a retail pharmacy location.

In conjunction with receiving a copayment (if any) from the member and dispensing the prescription drug to the member, the pharmacy submits a claim to the PBM for the prescription drug. After receiving the claim, the PBM (such as by using the benefit manager device 102) may perform certain adjudication operations including verifying eligibility for the member, identifying/reviewing an applicable formulary for the member to determine any appropriate copayment, coinsurance, and deductible for the prescription drug, and performing a drug utilization review (DUR) for the member. Further, the PBM may provide a response to the pharmacy (for example, the pharmacy system 100) following performance of at least some of the aforementioned operations.

As part of the adjudication, a plan sponsor (or the PBM on behalf of the plan sponsor) ultimately reimburses the pharmacy for filling the prescription drug when the prescription drug was successfully adjudicated. The aforementioned adjudication operations generally occur before the copayment is received and the prescription drug is dispensed. However in some instances, these operations may occur simultaneously, substantially simultaneously, or in a different order. In addition, more or fewer adjudication operations may be performed as at least part of the adjudication process.

The amount of reimbursement paid to the pharmacy by a plan sponsor and/or money paid by the member may be determined at least partially based on types of pharmacy networks in which the pharmacy is included. In some embodiments, the amount may also be determined based on other factors. For example, if the member pays the pharmacy for the prescription drug without using the prescription or drug benefit provided by the PBM, the amount of money paid by the member may be higher than when the member uses the prescription or drug benefit. In some embodiments, the amount of money received by the pharmacy for dispensing the prescription drug and for the prescription drug itself may be higher than when the member uses the prescription or drug benefit. Some or all of the foregoing operations may be performed by executing instructions stored in the benefit manager device 102 and/or an additional device.

Examples of the network 104 include a Global System for Mobile Communications (GSM) network, a code division multiple access (CDMA) network, 3rd Generation Partnership Project (3GPP), an Internet Protocol (IP) network, a Wireless Application Protocol (WAP) network, or an IEEE 802.11 standards network, as well as various combinations of the above networks. The network 104 may include an optical network. The network 104 may be a local area network or a global communication network, such as the Internet. In some embodiments, the network 104 may include a network dedicated to prescription orders: a prescribing network such as the electronic prescribing network operated by Surescripts of Arlington, Virginia.

Moreover, although the system shows a single network 104, multiple networks can be used. The multiple networks may communicate in series and/or parallel with each other to link the devices 102-110.

The pharmacy device 106 may be a device associated with a retail pharmacy location (e.g., an exclusive pharmacy location, a grocery store with a retail pharmacy, or a general sales store with a retail pharmacy) or other type of pharmacy location at which a member attempts to obtain a prescription. The pharmacy may use the pharmacy device 106 to submit the claim to the PBM for adjudication.

Additionally, or alternatively, in some embodiments, the pharmacy device 106 may enable information exchange between the pharmacy and the PBM. For example, this may allow the sharing of member information such as drug history that may allow the pharmacy to better service a member (for example, by providing more informed therapy consultation and drug interaction information). In some embodiments, the benefit manager device 102 may track prescription drug fulfillment and/or other information for users that are not members, or have not identified themselves as members, at the time (or in conjunction with the time) in which they seek to have a prescription filled at a pharmacy.

The pharmacy device 106 may include a pharmacy fulfillment device 112, an order processing device 114, and a pharmacy management device 116 in communication with each other directly and/or over the network 104. The order processing device 114 may receive information regarding filling prescriptions and may direct an order component to one or more devices of the pharmacy fulfillment device 112 at a pharmacy. The pharmacy fulfillment device 112 may fulfill, dispense, aggregate, and/or pack the order components of the prescription drugs in accordance with one or more prescription orders directed by the order processing device 114.

In general, the order processing device 114 is a device located within or otherwise associated with the pharmacy to enable the pharmacy fulfilment device 112 to fulfill a prescription and dispense prescription drugs. In some embodiments, the order processing device 114 may be an external order processing device separate from the pharmacy and in communication with other devices located within the pharmacy.

For example, the external order processing device may communicate with an internal pharmacy order processing device and/or other devices located within the system 100. In some embodiments, the external order processing device may have limited functionality (e.g., as operated by a user requesting fulfillment of a prescription drug), while the internal pharmacy order processing device may have greater functionality (e.g., as operated by a pharmacist).

The order processing device 114 may track the prescription order as it is fulfilled by the pharmacy fulfillment device 112. The prescription order may include one or more prescription drugs to be filled by the pharmacy. The order processing device 114 may make pharmacy routing decisions and/or order consolidation decisions for the particular prescription order. The pharmacy routing decisions include what device(s) in the pharmacy are responsible for filling or otherwise handling certain portions of the prescription order. The order consolidation decisions include whether portions of one prescription order or multiple prescription orders should be shipped together for a user or a user family. The order processing device 114 may also track and/or schedule literature or paperwork associated with each prescription order or multiple prescription orders that are being shipped together. In some embodiments, the order processing device 114 may operate in combination with the pharmacy management device 116.

The order processing device 114 may include circuitry, a processor, a memory to store data and instructions, and communication functionality. The order processing device 114 is dedicated to performing processes, methods, and/or instructions described in this application. Other types of electronic devices may also be used that are specifically configured to implement the processes, methods, and/or instructions described in further detail below.

In some embodiments, at least some functionality of the order processing device 114 may be included in the pharmacy management device 116. The order processing device 114 may be in a client-server relationship with the pharmacy management device 116, in a peer-to-peer relationship with the pharmacy management device 116, or in a different type of relationship with the pharmacy management device 116. The order processing device 114 and/or the pharmacy management device 116 may communicate directly (for example, such as by using a local storage) and/or through the network 104 (such as by using a cloud storage configuration, software as a service, etc.) with the storage device 110.

The storage device 110 may include: non-transitory storage (for example, memory, hard disk, CD-ROM, etc.) in communication with the benefit manager device 102 and/or the pharmacy device 106 directly and/or over the network 104. The non-transitory storage may store order data 118, member data 120, claims data 122, drug data 124, prescription data 126, and/or plan sponsor data 128. Further, the system 100 may include additional devices, which may communicate with each other directly or over the network 104.

The order data 118 may be related to a prescription order. The order data may include type of the prescription drug (for example, drug name and strength) and quantity of the prescription drug. The order data 118 may also include data used for completion of the prescription, such as prescription materials. In general, prescription materials include an electronic copy of information regarding the prescription drug for inclusion with or otherwise in conjunction with the fulfilled prescription. The prescription materials may include electronic information regarding drug interaction warnings, recommended usage, possible side effects, expiration date, date of prescribing, etc. The order data 118 may be used by a high-volume fulfillment center to fulfill a pharmacy order.

In some embodiments, the order data 118 includes verification information associated with fulfillment of the prescription in the pharmacy. For example, the order data 118 may include videos and/or images taken of (i) the prescription drug prior to dispensing, during dispensing, and/or after dispensing, (ii) the prescription container (for example, a prescription container and sealing lid, prescription packaging, etc.) used to contain the prescription drug prior to dispensing, during dispensing, and/or after dispensing, (iii) the packaging and/or packaging materials used to ship or otherwise deliver the prescription drug prior to dispensing, during dispensing, and/or after dispensing, and/or (iv) the fulfillment process within the pharmacy. Other types of verification information such as barcode data read from pallets, bins, trays, or carts used to transport prescriptions within the pharmacy may also be stored as order data 118.

The member data 120 includes information regarding the members associated with the PBM. The information stored as member data 120 may include personal information, personal health information, protected health information, etc. Examples of the member data 120 include name, address, telephone number, e-mail address, prescription drug history, etc. The member data 120 may include a plan sponsor identifier that identifies the plan sponsor associated with the member and/or a member identifier that identifies the member to the plan sponsor. The member data 120 may include a member identifier that identifies the plan sponsor associated with the user and/or a user identifier that identifies the user to the plan sponsor. The member data 120 may also include dispensation preferences such as type of label, type of cap, message preferences, language preferences, etc.

The member data 120 may be accessed by various devices in the pharmacy (for example, the high-volume fulfillment center, etc.) to obtain information used for fulfillment and shipping of prescription orders. In some embodiments, an external order processing device operated by or on behalf of a member may have access to at least a portion of the member data 120 for review, verification, or other purposes.

In some embodiments, the member data 120 may include information for persons who are users of the pharmacy but are not members in the pharmacy benefit plan being provided by the PBM. For example, these users may obtain drugs directly from the pharmacy, through a private label service offered by the pharmacy, the high-volume fulfillment center, or otherwise. In general, the use of the terms “member” and “user” may be used interchangeably.

The claims data 122 includes information regarding pharmacy claims adjudicated by the PBM under a drug benefit program provided by the PBM for one or more plan sponsors. In general, the claims data 122 includes an identification of the client that sponsors the drug benefit program under which the claim is made, and/or the member that purchased the prescription drug giving rise to the claim, the prescription drug that was filled by the pharmacy (e.g., the national drug code number, etc.), the dispensing date, generic indicator, generic product identifier (GPI) number, medication class, the cost of the prescription drug provided under the drug benefit program, the copayment/coinsurance amount, rebate information, and/or member eligibility, etc. Additional information may be included.

In some embodiments, other types of claims beyond prescription drug claims may be stored in the claims data 122. For example, medical claims, dental claims, wellness claims, or other types of health-care-related claims for members may be stored as a portion of the claims data 122.

In some embodiments, the claims data 122 includes claims that identify the members with whom the claims are associated. Additionally or alternatively, the claims data 122 may include claims that have been de-identified (that is, associated with a unique identifier but not with a particular, identifiable member).

The drug data 124 may include drug name (e.g., technical name and/or common name), other names by which the drug is known, active ingredients, an image of the drug (such as in pill form), etc. The drug data 124 may include information associated with a single medication or multiple medications.

The prescription data 126 may include information regarding prescriptions that may be issued by prescribers on behalf of users, who may be members of the pharmacy benefit plan—for example, to be filled by a pharmacy. Examples of the prescription data 126 include user names, medication or treatment (such as lab tests), dosing information, etc. The prescriptions may include electronic prescriptions or paper prescriptions that have been scanned. In some embodiments, the dosing information reflects a frequency of use (e.g., once a day, twice a day, before each meal, etc.) and a duration of use (e.g., a few days, a week, a few weeks, a month, etc.).

In some embodiments, the order data 118 may be linked to associated member data 120, claims data 122, drug data 124, and/or prescription data 126.

The plan sponsor data 128 includes information regarding the plan sponsors of the PBM. Examples of the plan sponsor data 128 include company name, company address, contact name, contact telephone number, contact e-mail address, etc.

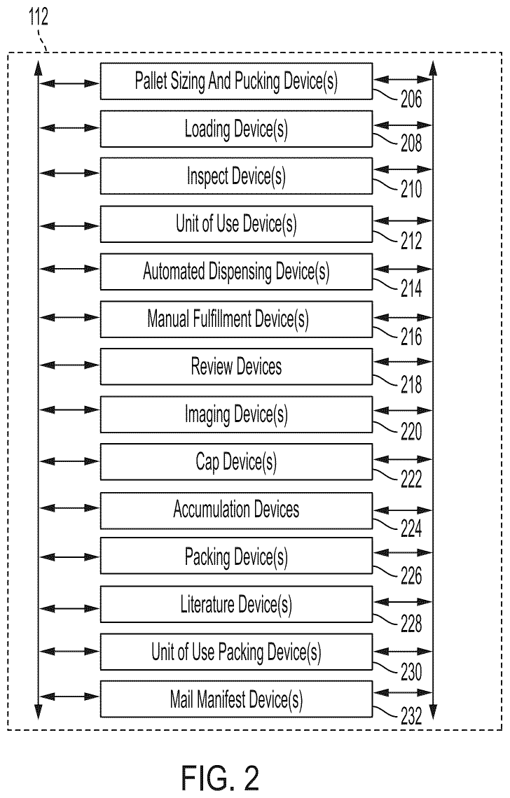

FIG. 2 illustrates the pharmacy fulfillment device 112 according to an example implementation. The pharmacy fulfillment device 112 may be used to process and fulfill prescriptions and prescription orders. After fulfillment, the fulfilled prescriptions are packed for shipping.

The pharmacy fulfillment device 112 may include devices in communication with the benefit manager device 102, the order processing device 114, and/or the storage device 110, directly or over the network 104. Specifically, the pharmacy fulfillment device 112 may include pallet sizing and pucking device(s) 206, loading device(s) 208, inspect device(s) 210, unit of use device(s) 212, automated dispensing device(s) 214, manual fulfillment device(s) 216, review devices 218, imaging device(s) 220, cap device(s) 222, accumulation devices 224, packing device(s) 226, literature device(s) 228, unit of use packing device(s) 230, and mail manifest device(s) 232. Further, the pharmacy fulfillment device 112 may include additional devices, which may communicate with each other directly or over the network 104.

In some embodiments, operations performed by one of these devices 206-232 may be performed sequentially, or in parallel with the operations of another device as may be coordinated by the order processing device 114. In some embodiments, the order processing device 114 tracks a prescription with the pharmacy based on operations performed by one or more of the devices 206-232.

In some embodiments, the pharmacy fulfillment device 112 may transport prescription drug containers, for example, among the devices 206-232 in the high-volume fulfillment center, by use of pallets. The pallet sizing and pucking device 206 may configure pucks in a pallet. A pallet may be a transport structure for a number of prescription containers, and may include a number of cavities. A puck may be placed in one or more than one of the cavities in a pallet by the pallet sizing and pucking device 206. The puck may include a receptacle sized and shaped to receive a prescription container. Such containers may be supported by the pucks during carriage in the pallet. Different pucks may have differently sized and shaped receptacles to accommodate containers of differing sizes, as may be appropriate for different prescriptions.

The arrangement of pucks in a pallet may be determined by the order processing device 114 based on prescriptions that the order processing device 114 decides to launch. The arrangement logic may be implemented directly in the pallet sizing and pucking device 206. Once a prescription is set to be launched, a puck suitable for the appropriate size of container for that prescription may be positioned in a pallet by a robotic arm or pickers. The pallet sizing and pucking device 206 may launch a pallet once pucks have been configured in the pallet.

The loading device 208 may load prescription containers into the pucks on a pallet by a robotic arm, a pick and place mechanism (also referred to as pickers), etc. In some embodiments, the loading device 208 has robotic arms or pickers to grasp a prescription container and move it to and from a pallet or a puck. The loading device 208 may also print a label that is appropriate for a container that is to be loaded onto the pallet, and apply the label to the container. The pallet may be located on a conveyor assembly during these operations (e.g., at the high-volume fulfillment center, etc.).

The inspect device 210 may verify that containers in a pallet are correctly labeled and in the correct spot on the pallet. The inspect device 210 may scan the label on one or more containers on the pallet. Labels of containers may be scanned or imaged in full or in part by the inspect device 210. Such imaging may occur after the container has been lifted out of its puck by a robotic arm, picker, etc., or may be otherwise scanned or imaged while retained in the puck. In some embodiments, images and/or video captured by the inspect device 210 may be stored in the storage device 110 as order data 118.

The unit of use device 212 may temporarily store, monitor, label, and/or dispense unit of use products. In general, unit of use products are prescription drug products that may be delivered to a user or member without being repackaged at the pharmacy. These products may include pills in a container, pills in a blister pack, inhalers, etc. Prescription drug products dispensed by the unit of use device 212 may be packaged individually or collectively for shipping, or may be shipped in combination with other prescription drugs dispensed by other devices in the high-volume fulfillment center.

At least some of the operations of the devices 206-232 may be directed by the order processing device 114. For example, the manual fulfillment device 216, the review device 218, the automated dispensing device 214, and/or the packing device 226, etc. may receive instructions provided by the order processing device 114.

The automated dispensing device 214 may include one or more devices that dispense prescription drugs or pharmaceuticals into prescription containers in accordance with one or multiple prescription orders. In general, the automated dispensing device 214 may include mechanical and electronic components with, in some embodiments, software and/or logic to facilitate pharmaceutical dispensing that would otherwise be performed in a manual fashion by a pharmacist and/or pharmacist technician. For example, the automated dispensing device 214 may include high-volume fillers that fill a number of prescription drug types at a rapid rate and blister pack machines that dispense and pack drugs into a blister pack. Prescription drugs dispensed by the automated dispensing devices 214 may be packaged individually or collectively for shipping, or may be shipped in combination with other prescription drugs dispensed by other devices in the high-volume fulfillment center.

The manual fulfillment device 216 controls how prescriptions are manually fulfilled. For example, the manual fulfillment device 216 may receive or obtain a container and enable fulfillment of the container by a pharmacist or pharmacy technician. In some embodiments, the manual fulfillment device 216 provides the filled container to another device in the pharmacy fulfillment devices 112 to be joined with other containers in a prescription order for a user or member.

In general, manual fulfillment may include operations at least partially performed by a pharmacist or a pharmacy technician. For example, a person may retrieve a supply of the prescribed drug, may make an observation, may count out a prescribed quantity of drugs and place them into a prescription container, etc. Some portions of the manual fulfillment process may be automated by use of a machine. For example, counting of capsules, tablets, or pills may be at least partially automated (such as through use of a pill counter). Prescription drugs dispensed by the manual fulfillment device 216 may be packaged individually or collectively for shipping, or may be shipped in combination with other prescription drugs dispensed by other devices in the high-volume fulfillment center.

The review device 218 may process prescription containers to be reviewed by a pharmacist for proper pill count, exception handling, prescription verification, etc. Fulfilled prescriptions may be manually reviewed and/or verified by a pharmacist, as may be required by state or local law. A pharmacist or other licensed pharmacy person who may dispense certain drugs in compliance with local and/or other laws may operate the review device 218 and visually inspect a prescription container that has been filled with a prescription drug. The pharmacist may review, verify, and/or evaluate drug quantity, drug strength, and/or drug interaction concerns, or otherwise perform pharmacist services. The pharmacist may also handle containers which have been flagged as an exception, such as containers with unreadable labels, containers for which the associated prescription order has been canceled, containers with defects, etc. In an example, the manual review can be performed at a manual review station.

The imaging device 220 may image containers once they have been filled with pharmaceuticals. The imaging device 220 may measure a fill height of the pharmaceuticals in the container based on the obtained image to determine if the container is filled to the correct height given the type of pharmaceutical and the number of pills in the prescription. Images of the pills in the container may also be obtained to detect the size of the pills themselves and markings thereon. The images may be transmitted to the order processing device 114 and/or stored in the storage device 110 as part of the order data 118.

The cap device 222 may be used to cap or otherwise seal a prescription container. In some embodiments, the cap device 222 may secure a prescription container with a type of cap in accordance with a user preference (e.g., a preference regarding child resistance, etc.), a plan sponsor preference, a prescriber preference, etc. The cap device 222 may also etch a message into the cap, although this process may be performed by a subsequent device in the high-volume fulfillment center.

The accumulation device 224 accumulates various containers of prescription drugs in a prescription order. The accumulation device 224 may accumulate prescription containers from various devices or areas of the pharmacy. For example, the accumulation device 224 may accumulate prescription containers from the unit of use device 212, the automated dispensing device 214, the manual fulfillment device 216, and the review device 218. The accumulation device 224 may be used to group the prescription containers prior to shipment to the member.

The literature device 228 prints, or otherwise generates, literature to include with each prescription drug order. The literature may be printed on multiple sheets of substrates, such as paper, coated paper, printable polymers, or combinations of the above substrates. The literature printed by the literature device 228 may include information required to accompany the prescription drugs included in a prescription order, other information related to prescription drugs in the order, financial information associated with the order (for example, an invoice or an account statement), etc.

In some embodiments, the literature device 228 folds or otherwise prepares the literature for inclusion with a prescription drug order (e.g., in a shipping container). In some embodiments, the literature device 228 prints the literature and is separate from another device that prepares the printed literature for inclusion with a prescription order.

The packing device 226 packages the prescription order in preparation for shipping the order. The packing device 226 may box, bag, or otherwise package the fulfilled prescription order for delivery. The packing device 226 may further place inserts (e.g., literature or other papers, etc.) into the packaging received from the literature device 228. For example, bulk prescription orders may be shipped in a box, while other prescription orders may be shipped in a bag, which may be a wrap seal bag.

The packing device 226 may label the box or bag with an address and a recipient's name. The label may be printed and affixed to the bag or box, be printed directly onto the bag or box, or otherwise associated with the bag or box. The packing device 226 may sort the box or bag for mailing in an efficient manner (e.g., sort by delivery address, etc.). The packing device 226 may include ice or temperature sensitive elements for prescriptions that are to be kept within a temperature range during shipping (for example, this may be necessary in order to retain efficacy). The ultimate package may then be shipped through postal mail, through a mail order delivery service that ships via ground and/or air (e.g., UPS, FEDEX, or DHL, etc.), through a delivery service, through a locker box at a shipping site (e.g., AMAZON locker or a PO Box, etc.), or otherwise.

The unit of use packing device 230 packages a unit of use prescription order in preparation for shipping the order. The unit of use packing device 230 may include manual scanning of containers to be bagged for shipping to verify each container in the order. In some embodiments, the manual scanning may be performed at a manual scanning station. The pharmacy fulfillment device 112 may also include a mail manifest device 232 to print mailing labels used by the packing device 226 and may print shipping manifests and packing lists.

While the pharmacy fulfillment device 112 in FIG. 2 is shown to include single devices 206-232, multiple devices may be used. When multiple devices are present, the multiple devices may be of the same device type or models, or may be a different device type or model. The types of devices 206-232 shown in FIG. 2 are example devices. In other configurations of the system 100, lesser, additional, or different types of devices may be included.

Moreover, multiple devices may share processing and/or memory resources. The devices 206-232 may be located in the same area or in different locations. For example, the devices 206-232 may be located in a building or set of adjoining buildings. The devices 206-232 may be interconnected (such as by conveyors), networked, and/or otherwise in contact with one another or integrated with one another (e.g., at the high-volume fulfillment center, etc.). In addition, the functionality of a device may be split among a number of discrete devices and/or combined with other devices.

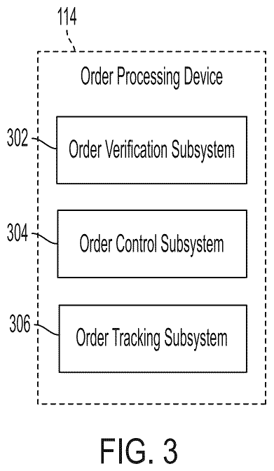

FIG. 3 illustrates the order processing device 114 according to some embodiments. The order processing device 114 may be used by one or more operators to generate prescription orders, make routing decisions, make prescription order consolidation decisions, track literature with the system 100, and/or view order status and other order related information. For example, the prescription order may include order components.

The order processing device 114 may receive instructions to fulfill an order without operator intervention. An order component may include a prescription drug fulfilled by use of a container through the system 100. The order processing device 114 may include an order verification subsystem 302, an order control subsystem 304, and/or an order tracking subsystem 306. Other subsystems may also be included in the order processing device 114.

The order verification subsystem 302 may communicate with the benefit manager device 102 to verify the eligibility of the member and review the formulary to determine appropriate copayment, coinsurance, and deductible for the prescription drug and/or perform a DUR (drug utilization review). Other communications between the order verification subsystem 302 and the benefit manager device 102 may be performed for a variety of purposes.

The order control subsystem 304 controls various movements of the containers and/or pallets along with various filling functions during their progression through the system 100. In some embodiments, the order control subsystem 304 may identify the prescribed drug in one or more than one prescription orders as capable of being fulfilled by the automated dispensing device 214. The order control subsystem 304 may determine which prescriptions are to be launched and may determine that a pallet of automated-fill containers is to be launched.

The order control subsystem 304 may determine that an automated-fill prescription of a specific pharmaceutical is to be launched and may examine a queue of orders awaiting fulfillment for other prescription orders, which will be filled with the same pharmaceutical. The order control subsystem 304 may then launch orders with similar automated-fill pharmaceutical needs together in a pallet to the automated dispensing device 214. As the devices 206-232 may be interconnected by a system of conveyors or other container movement systems, the order control subsystem 304 may control various conveyors: for example, to deliver the pallet from the loading device 208 to the manual fulfillment device 216 from the literature device 228, paperwork as needed to fill the prescription.

The order tracking subsystem 306 may track a prescription order during its progress toward fulfillment. The order tracking subsystem 306 may track, record, and/or update order history, order status, and the like. The order tracking subsystem 306 may store data locally (for example, in a memory) or as a portion of the order data 118 stored in the storage device 110.

In some embodiments, the system 100 may include one or more computing devices 108, as is generally illustrated in FIG. 1B. The computing device 108 may include any suitable computing device, such as a mobile computing device, a desktop computing device, a laptop computing device, a server computing device, other suitable computing device, or a combination thereof. The computing device 108 may be used by a user accessing the pharmacy associated with the system 100, as described. Additionally, or alternatively, the computing device 108 may be configured to identify an optimum or substantially optimum combination of data objects, as described.

The computing device 108 may include a processor 130 configured to control the overall operation of computing device 108. The processor 130 may include any suitable processor, such as those described herein. The computing device 108 may also include a user input device 132 that is configured to receive input from a user of the computing device 108 and to communicate signals representing the input received from the user to the processor 130. For example, the user input device 132 may include a button, keypad, dial, touch screen, audio input interface, visual/image capture input interface, input in the form of sensor data, etc.

The computing device 108 may include a display 136 that may be controlled by the processor 130 to display information to the user. A data bus 138 may be configured to facilitate data transfer between, at least, a storage device 140 and the processor 130. The computing device 108 may also include a network interface 142 configured to couple or connect the computing device 108 to various other computing devices or network devices via a network connection, such as a wired or wireless connection, such as the network 104. In some embodiments, the network interface 142 includes a wireless transceiver.

The storage device 140 may include a single disk or a plurality of disks (e.g., hard drives), one or more solid-state drives, one or more hybrid hard drives, and the like. The storage device 140 may include a storage management module that manages one or more partitions within the storage device 140. In some embodiments, storage device 140 may include flash memory, semiconductor (solid state) memory or the like. The computing device 108 may also include a memory 144. The memory 144 may include Random Access Memory (RAM), a Read-Only Memory (ROM), or a combination thereof. The memory 144 may store programs, utilities, or processes to be executed in by the processor 130. The memory 144 may provide volatile data storage, and stores instructions related to the operation of the computing device 108. In some embodiments, the processor 130 may be configured to execute instructions stored on the memory 144 to, at least, perform various aspects of the systems and methods described herein.

In some embodiments, the computing device 108 may include an artificial intelligence engine 146 configured to use one or more machine learning models 148 configured to perform at least some aspects of the systems and methods described herein. The artificial intelligence engine 146 may include any suitable artificial intelligence engine and may be disposed on computing device 108 or remotely located from the computing device 108, such as in a cloud computing device or other suitable remotely located computing device. The computing device 108 may include a training engine capable of generating the one or more machine learning models 148. The one or more machine learning models 148 may be trained using any suitable data, including those described herein. Additionally, or alternatively, the one or more machine learning models 148 may be iteratively trained (e.g., subsequent to an initiate training) using output from the one or more machine learning models 148 (e.g., as feedback, which may or may not include input from a user indicating accuracy of one or more predictions associated with the output of the one or more machine learning models 148).

In some embodiments, the computing device 108 may be configured to receive, responsive to the user filling at least one prescription, a prescription notification indicating information corresponding to the at least one prescription and information corresponding to the user. The information corresponding to the at least one prescription may include at least prescription identification information, dosing information, other suitable information, and the like. The information corresponding to the user may include at least user statistical information, user contact information, other suitable information, and the like.

The computing device 108 may identify, based on the prescription notification, at least one health condition of the user. The computing device 108 may, in response to the at least one health condition corresponding to at least one health condition of a plurality of predetermined health conditions, generate a message including instructions for downloading a user application (e.g., at a computing device associated with the user). The plurality of predetermined health conditions may include at least one of at least one behavioral health condition, at least one musculoskeletal health condition, and/or at least one other health condition. The at least one behavioral health condition may correspond to at least one of depression, generalized anxiety, bipolar disorder, at least one other behavioral health condition, and/or any other suitable behavioral health condition. The at least one musculoskeletal health condition may correspond to at least one of back pain, neck pain, joint pain, at least one other musculoskeletal health condition, and/or any other suitable musculoskeletal health condition. The at least one other health condition corresponds to at least one of cancer, diabetes, heart failure, heart disease, chronic obstructive pulmonary disease, covid 19, at least one other health condition, and/or any other suitable health condition.

The computing device 108 may communicate (e.g., via the network interface 142 or other suitable interface), to a user account associated with the user, the message. The user account associated with the user may include an electronic mail account, a pharmacy account, a health insurance account, at least one other user account, and/or the like. The computing device 108 may, in response to an indication that the user initiated the user application, provide, at an application setup interface associated with the user application (e.g., provided at the computing device associated with the user), a plurality of data gathering queries. The data gathering queries may include at least one questions corresponding to a mental or emotional state of the user, a pain level of the user, an energy level of the user, and/or the like. Additionally, or alternatively, the data gathering queries may include one or more questions corresponding to the age of the user, the family history of the user, the location of the user, and/or the like. Additionally, or alternatively, the data gathering queries may correspond to a level of financial comfort of the user, a level of comfort of the user with insurance benefits, demographics of the user, technology access of the user, and/or the like. It should be understood that the data gathering queries may correspond to any suitable information to be gathered from the user.

The computing device 108 may store, in a database or other suitable data store (e.g., such as the storage device 110, the storage device 140, and/or any other suitable device), user responses to the data gathering queries. The computing device 108 may generate, using the user responses, a data structure corresponding to the user. The computing device 108 may generate, based on the data structure corresponding to the user, a personalized experience interface, such as the personalize experience interface 500. In some embodiments, the personalized experience interface 500 may comprise a portable health record comprising the health record of the user. The personalized experience interface 500 may include one or more personalized components that include at least a daily interaction component, a relevant resource component, a dynamic selection component, and/or the like. The dynamic selection component may include at least one general selection and/or, responsive to the at least one health condition, at least one health condition specific component.

The computing device 108 may provide, at a display of the computing device associated with the user or other suitable display, the personalized experience interface 500. The personalized experience interface 500 may correspond to a user avatar that models a biological identity of the user. The user avatar may correspond to personalized, intelligence associated with a predictive healthcare driven model and may be based on a data driven, dynamic patient survey. The computing device 108 may provide, at a metaverse (e.g., a three-dimensional virtual environment), access to at least one three-dimensional environment feature using the user avatar. The at least one three-dimensional environment feature may include a virtual healthcare provider office, a virtual healthcare provider, at least one other three-dimensional environment feature, and/or the like. The virtual healthcare provider may include an avatar corresponding to a healthcare provider (e.g., in the real-world), an avatar corresponding to an artificially intelligent healthcare provider (e.g., a virtual healthcare provider driving my artificial intelligence), and/or the like.

The computing device 108 may identify, using a first set of user responses, a healthcare provider pool. The computing device 108 may adjust at least some of the data gathering queries based on the first set of user responses. The s computing device 108 may identify, responsive to a second set of user responses corresponding to at least some of the adjusted data gathering queries, a subset of healthcare providers of the healthcare provider pool that match with the user.

In some embodiments, the personalized experience interface 500 may include, at least, information corresponding to each of the subset of healthcare providers. The computing device 108 may schedule, responsive to a selection of at least one healthcare provider of the subset of healthcare providers by the user using the personalized experience interface 500, at least one appointment, for the user, with the selected at least one healthcare provider.

The computing device 108 may receive feedback from the user corresponding to at least one of a component of the personalized experience interface 500, an appointment with the selected at least one healthcare provider and/or any other suitable aspect of the systems and methods described herein. The computing device 108 may refine, responsive to the feedback, the data gathering queries, the personalized experience interface 500, and/or any other suitable feature. The computing device 108 may identify, based on the feedback, at least one other healthcare provider from the healthcare provider pool.

The computing device 108 may select, using the data structure corresponding to the user, at least one suggestive aspect of the personalized experience interface 500. The computing device 108 may provide, at the personalized experience interface 500, the at least one suggestive aspect. The at least one suggestive aspect may include at least one of a color, a dynamic background, at least one other suggestive aspect, and/or the like. Additionally, or alternatively, the at least one suggestive aspect may be configured to provide, responsive to the user engaging with the at least one suggestive aspect, at least one of a physical response of the user and a mental response of the user.

The computing device 108 may generate or refine one or more of at least one aspect of the data gathering queries, at least one aspect of the personalized experience interface 500, and/or the like, using at least one machine learning model, such as the machine learning model 148 or other suitable machine learning model. The machine learning model 148 may be configured to use at least some of the user responses and at least some responses provided by other users to dynamically generate or refine the at least one of at least one aspect of the data gathering queries, at least one aspect of the personalized experience interface 500, and/or any other suitable feature or aspect of the systems and methods described herein.

In some embodiments, the computing device 108 and/or the system 100 may perform the methods described herein. However, the methods described herein as performed by the computing device 108 and/or the system 100 are not meant to be limiting, and any type of software executed on a computing device or a combination of various computing devices can perform the methods described herein without departing from the scope of this disclosure.

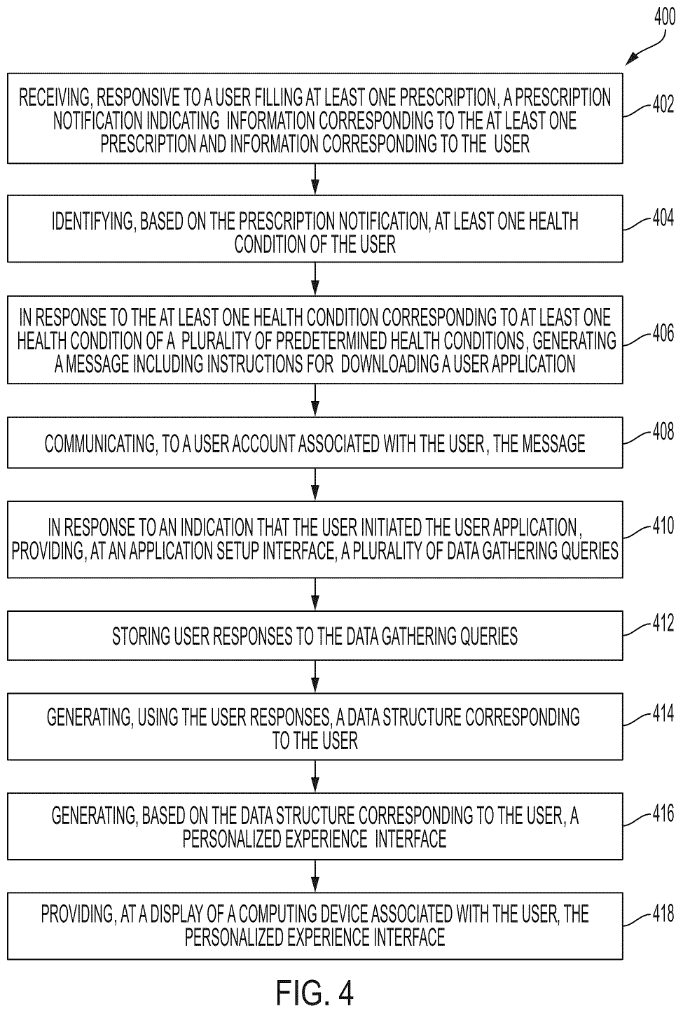

FIG. 4 is a flow diagram generally illustrating personalized healthcare experience method 400 according to the principles of the present disclosure. At 402, the method 400 receives, responsive to a user filling at least one prescription, a prescription notification indicating information corresponding to the at least one prescription and information corresponding to the user. For example, the computing device 108 may receive, responsive to the user filling the at least one prescription, the prescription notification.

At 404, the method 40 identifies, based on the prescription notification, at least one health condition of the user. For example, the computing device 108 may identify, based on the prescription notification, the at least one health condition of the user. The at least one health condition of the user may include a behavioral health condition or other suitable condition.

At 406, the method 400, in response to the at least one health condition corresponding to at least one health condition of a plurality of predetermined health conditions, generates a message including instructions for downloading a user application. For example, the computing device 108 may generate, in response to the at least one health condition corresponding to at least one health condition of a plurality of predetermined health conditions, the message including instructions for downloading the user application.

At 408, the method 400 communicates, to a user account associated with the user, the message. For example, the computing device 108 may communicate the message to the user account associated with the user.

At 410, the method 400, in response to an indication that the user initiated the user application, provides, at an application setup interface, a plurality of data gathering queries. For example, the computing device 108 may, in response to the indication that the user initiated the user application, provide, at the application setup interface, the plurality of data queries.

At 412, the method 400 stores user responses to the data gathering queries. For example, the computing device 108 may store the user responses to the data gathering queries.

At 414, the method 400 generates, using the user responses, a data structure corresponding to the user. For example, the computing device 108 may generate, using the user responses, the data structure corresponding to the user.

At 416, the method 400 generates, based on the data structure corresponding to the user, a personalized experience interface. For example, the computing device 108 may generate, based on the data structure corresponding to the user, the personalized experience interface 500. The personalized experience interface 500 may be configured to provide behavioral health components, such as sleep trackers, exercise trackers, daily mental health tips, and the like. Additionally, or alternatively, the personalized experience interface 500 may provide daily queries to the user (e.g., requesting the user provide input indicating how the user is feeling, how the user has been felt over a period of time, what the user's mental state is, and/or various other information associated with the user). The computing device 108 may use input provided by the user to take one or more actions. For example, if the user provides input (e.g., based on a signal response and/or a combination of multiple responses) that indicates a certain behavioral concern (e.g., self-harm or other suitable behavioral concern), the computing device 108 may generate an alert to a human or artificial intelligence agent to contact the user and/or a healthcare provider.

At 418, the method 400 provides, at a display of a computing device associated with the user, the personalized experience interface. For example, the computing device 108 may provide, at the display of the computing device associated with the user, the personal experience interface 500.

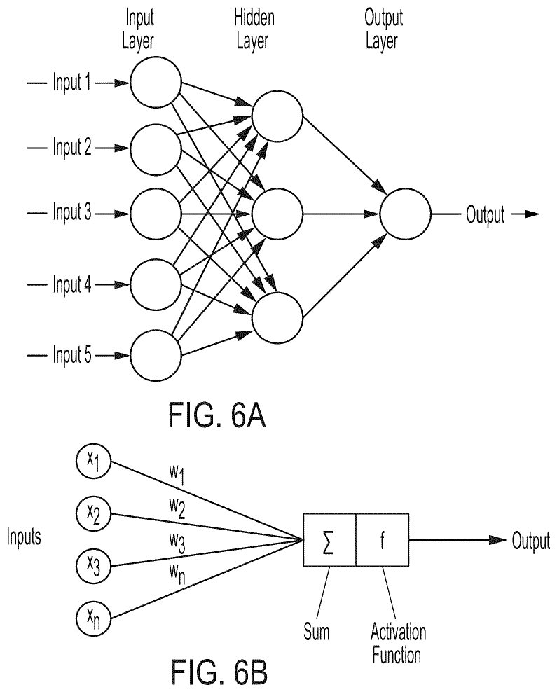

FIG. 6A shows a fully connected neural network (e.g., which may be associated with the machine learning model 148), where each neuron in a given layer is connected to each neuron in a next layer. In the input layer, each input node is associated with a numerical value, which can be any real number. The neural network may be used to perform at least some of the features of the systems and methods described herein. In each layer, each connection that departs from an input node has a weight associated with it, which can also be any real number (see FIG. 6B). In the input layer, the number of neurons equals number of features (columns) in a dataset. The output layer may have multiple continuous outputs.

The layers between the input and output layers are hidden layers. The number of hidden layers can be one or more (one hidden layer may be sufficient for most applications). A neural network with no hidden layers can represent linear separable functions or decisions. A neural network with one hidden layer can perform continuous mapping from one finite space to another. A neural network with two hidden layers can approximate any smooth mapping to any accuracy.

The number of neurons can be optimized. At the beginning of training, a network configuration is more likely to have excess nodes. Some of the nodes may be removed from the network during training that would not noticeably affect network performance. For example, nodes with weights approaching zero after training can be removed (this process is called pruning). The number of neurons can cause under-fitting (inability to adequately capture signals in dataset) or over-fitting (insufficient information to train all neurons; network performs well on training dataset but not on test dataset).

Various methods and criteria can be used to measure performance of a neural network model. For example, root mean squared error (RMSE) measures the average distance between observed values and model predictions. Coefficient of Determination (R2) measures correlation (not accuracy) between observed and predicted outcomes. This method may not be reliable if the data has a large variance. Other performance measures include irreducible noise, model bias, and model variance. A high model bias for a model indicates that the model is not able to capture true relationship between predictors and the outcome. Model variance may indicate whether a model is stable (a slight perturbation in the data will significantly change the model fit). The neural network can receive inputs, e.g., vectors, that can be used to generate models that can be used with provider matching, risk model processing, or both, as described herein.

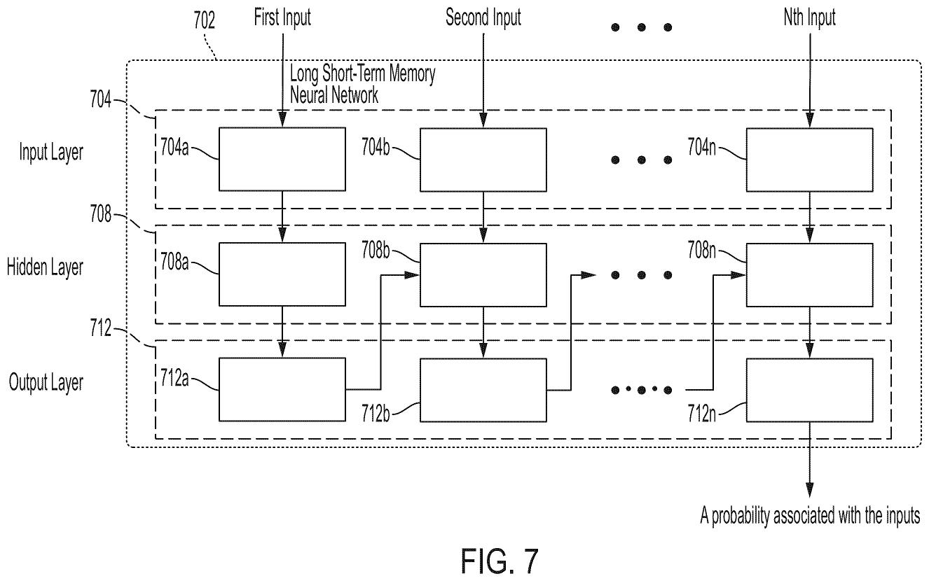

FIG. 7 illustrates an example of a long short-term memory (LSTM) neural network 702 used to generate models such as those described above, using machine learning techniques. The LSTM neural network 702 may be used to implement a machine learning model, such as the machine learning model 148 or other suitable machine learning model, and, in some embodiments, may use other types of machine learning networks. The LSTM network 702 may be used to implement at least some of the features of the systems and methods described herein. The LSTM neural network 702 includes an input layer 704, a hidden layer 708, and an output layer 712. The input layer 704 includes inputs 704a, 704b . . . 704n. The hidden layer 708 includes neurons 708a, 708b . . . 708n. The output layer 712 includes outputs 712a, 712b . . . 712n.

Each neuron of the hidden layer 708 receives an input, such as those described with respect to FIGS. 1-5, from the input layer 704 and outputs a value to the corresponding output in the output layer 712 (e.g., including, but not limited to, one or more outputs corresponding to the generation or refinement of one or more of at least one aspect of the data gathering queries, at least one aspect of the personalized experience interface 500, and/or the like, as described). For example, the neuron 708a receives an input from the input 704a and outputs a value to the output 712a. Each neuron, other than the neuron 708a, also receives an output of a previous neuron as an input. For example, the neuron 708b receives inputs from the input 704b and the output 712a. In this way the output of each neuron is fed forward to the next neuron in the hidden layer 708. The last output 712n in the output layer 712 outputs a probability associated with the inputs 704a-704n. Although the input layer 704, the hidden layer 708, and the output layer 712 are depicted as each including three elements, each layer may contain any number of elements.

In some embodiments, each layer of the LSTM neural network 702 must include the same number of elements as each of the other layers of the LSTM neural network 702. In some embodiments, a convolutional neural network may be implemented. Similar to LSTM neural networks, convolutional neural networks include an input layer, a hidden layer, and an output layer. However, in a convolutional neural network, the output layer includes one fewer output than the number of neurons in the hidden layer and each neuron is connected to each output. Additionally, each input in the input layer is connected to each neuron in the hidden layer. In other words, input 404a is connected to each of neurons 708a, 708b . . . 708n.

In some embodiments, each input node in the input layer may be associated with a numerical value, which can be any real number. In each layer, each connection that departs from an input node has a weight associated with it, which can also be any real number. In the input layer, the number of neurons equals number of features (columns) in a dataset. The output layer may have multiple continuous outputs.

As mentioned above, the layers between the input and output layers are hidden layers. The number of hidden layers can be one or more (one hidden layer may be sufficient for many applications). A neural network with no hidden layers can represent linear separable functions or decisions. A neural network with one hidden layer can perform continuous mapping from one finite space to another. A neural network with two hidden layers can approximate any smooth mapping to any accuracy. The neural network of FIG. 7 can receive inputs, e.g., vectors, that can be used to generate models that can be used with provider matching, risk model processing, or both, as described herein.

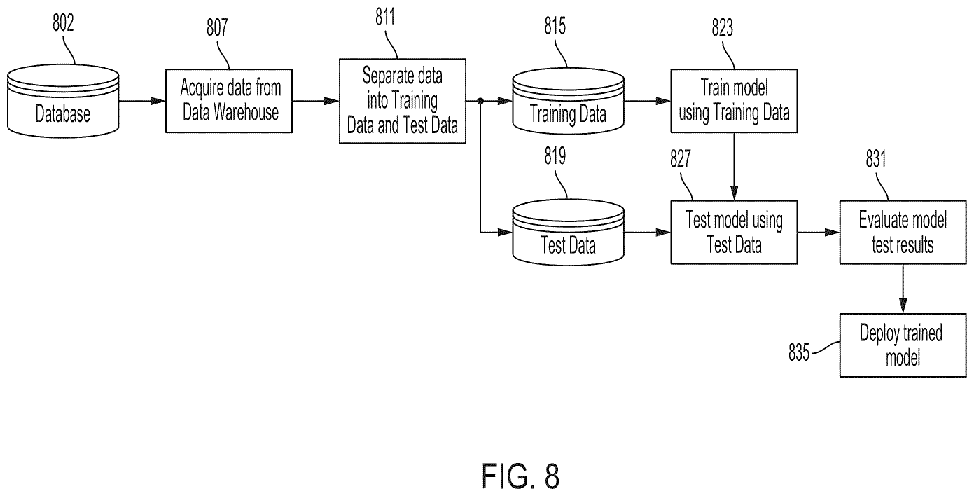

FIG. 8 illustrates an example process for generating a machine learning model, including, but not limited to, the machine learning model 148. At 807, control obtains data from a database 802 (e.g., a data warehouse), such as the database 402. The data may include any suitable data for developing machine learning models, such as the machine learning model 148 and/or any other suitable machine learning models.

At 811, control separates the data obtained from the database 802 into training data 815 and test data 819. The training data 815 is used to train the model at 823, and the test data 819 is used to test the model at 827. Typically, the set of training data 815 is selected to be larger than the set of test data 819, depending on the desired model development parameters. For example, the training data 815 may include about seventy percent of the data acquired from the database 802, about eighty percent of the data, about ninety percent, etc. The remaining thirty percent, twenty percent, or ten percent, is then used as the test data 819.

Separating a portion of the acquired data as test data 819 allows for testing of the trained model against actual output data, to facilitate more accurate training and development of the model at 823 and 827. The model may be trained at 823 using any suitable machine learning model techniques, including those described herein, such as random forest, generalized linear models, decision tree, and neural networks.

At 831, control evaluates the model test results. For example, the trained model may be tested at 827 using the test data 819, and the results of the output data from the tested model may be compared to actual outputs of the test data 819, to determine a level of accuracy. The model results may be evaluated using any suitable machine learning model analysis, such as the example techniques described further below.

After evaluating the model test results at 831, the model may be deployed at 835 if the model test results are satisfactory. Deploying the model may include using the model to make predictions for a large-scale input dataset with unknown outputs. If the evaluation of the model test results at 831 is unsatisfactory, the model may be developed further using different parameters, using different modeling techniques, using other model types, etc. The machine learning model method of FIG. 8 can receive inputs, e.g., vectors, that can be used to generate models that can be used with provider matching, risk model processing, or both, as described herein.

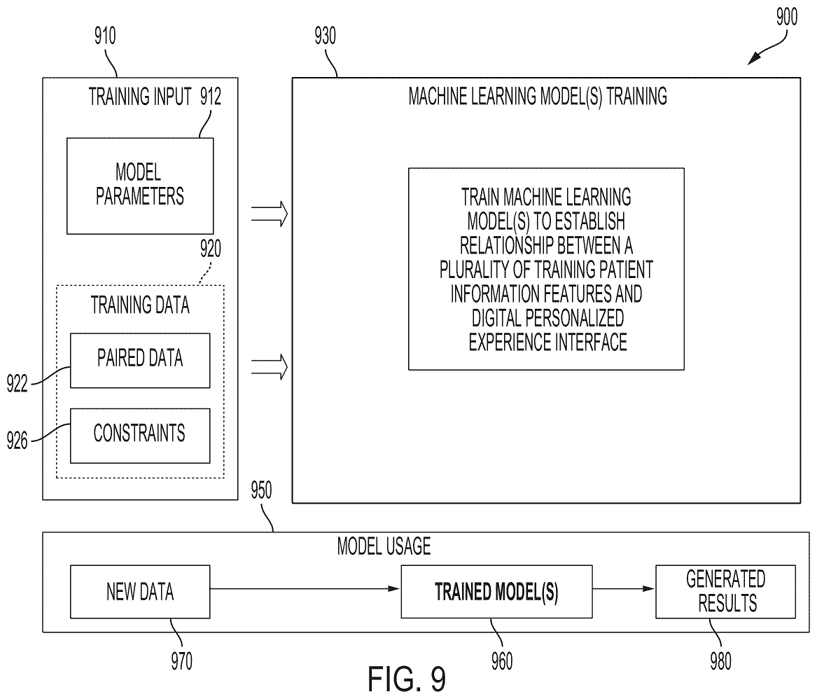

FIG. 9 is a block diagram of an example an interactive digital personalized experience interface system 600 that may be deployed within the system of FIG. 1, according to some embodiments. Training input 910 includes model parameters 912 and training data 920 (e.g., training data related to user interfaces, behavioral health data, and/or the like) which may include paired training data sets 922 (e.g., input-output training pairs) and constraints 926. Model parameters 912 stores or provides the parameters or coefficients of corresponding ones of machine learning models. During training, these parameters 912 are adapted based on the input-output training pairs of the training data sets 922. After the parameters 912 are adapted (after training), the parameters are used by trained models 960 to implement the trained machine learning models on a new set of data 970.

Training data 920 includes constraints 926 which may define the constraints of a given patient information features. The paired training data sets 922 may include sets of input-output pairs, such as a pairs of a plurality of training patient information features and types of service of care associated with the training patient information features. Some components of training input 910 may be stored separately at a different off-site facility or facilities than other components.

Machine learning model(s) training 930 trains one or more machine learning techniques based on the sets of input-output pairs of paired training data sets 922. For example, the model training 930 may train the machine learning model parameters 912 by minimizing a loss function based on one or more ground-truth type of service of care. The machine learning model can include any one or combination of classifiers or neural networks, such as an artificial neural network, a convolutional neural network, an adversarial network, a generative adversarial network, a deep feed forward network, a radial basis network, a recurrent neural network, a long/short term memory network, a gated recurrent unit, an auto encoder, a variational autoencoder, a denoising autoencoder, a sparse autoencoder, a Markov chain, a Hopfield network, a Boltzmann machine, a restricted Boltzmann machine, a deep belief network, a deep convolutional network, a deconvolutional network, a deep convolutional inverse graphics network, a liquid state machine, an extreme learning machine, an echo state network, a deep residual network, a Kohonen network, a support vector machine, a neural Turing machine, and the like.

Particularly, the machine learning model can be applied to a training plurality of patient information features to estimate or generate a prediction of a type of service of care. In some embodiments, a derivative of a loss function is computed based on a comparison of the estimated type of service of care and the ground truth type of service of care associated with the training patient information features and parameters of the machine learning model are updated based on the computed derivative of the loss function.

In some embodiments, the machine learning model receives a batch of training data that includes a first set of the plurality of training patient information features together with a ground-truth type of service of care associated with the first set of the plurality of training patient information features. The machine learning model generates a feature vector based on the first set of the plurality of training patient information features and generates a prediction of one or more types of service of care. The prediction is compared with the ground truth type of service of care and parameters of the machine learning model are updated based on the comparison.

The result of minimizing the loss function for multiple sets of training data trains, adapts, or optimizes the model parameters 912 of the corresponding machine learning models. In this way, the machine learning model is trained to establish a relationship between a plurality of training patient information features and types of service of care.

After the machine learning model is trained, new data 970, including one or more patient information features are received. The trained machine learning technique may be applied to the new data 970 to generate results 980 including a prediction of a service of care type to recommend. The selection or recommendation made by the system 156 can be represented in a graphical user interface that depicts each of a plurality of different types of service of care or other patient interactive interface.

In some embodiments, a method for providing an interactive digital personalized experience interface includes receiving, responsive to a user filling at least one prescription, a prescription notification indicating information corresponding to the at least one prescription and information corresponding to the user. The method also includes identifying, based on the prescription notification, at least one health condition of the user. The method also includes, in response to the at least one health condition corresponding to at least one health condition of a plurality of predetermined health conditions, generating a message including instructions for downloading a user application. The method also includes communicating, to a user account associated with the user, the message and, in response to an indication that the user initiated the user application, providing, at an application setup interface, a plurality of data gathering queries. The method also includes storing user responses to the data gathering queries and generating, using the user responses, a data structure corresponding to the user. The method also includes generating, based on the data structure corresponding to the user, a personalized experience interface, and providing, at a display of a computing device associated with the user, the personalized experience interface.

In some embodiments, the information corresponding to the at least one prescription includes at least prescription identification information and dosing information. In some embodiments, the information corresponding to the user includes at least user statistical information and user contact information. In some embodiments, the plurality of predetermined health conditions include at least one of at least one behavioral health condition, at least one musculoskeletal health condition, and at least one other health condition. In some embodiments, the at least one behavioral health condition corresponds to at least one of depression, generalized anxiety, bipolar disorder, and at least one other behavioral health condition. In some embodiments, the at least one musculoskeletal health condition corresponds to at least one of back pain, neck pain, joint pain, and at least one other musculoskeletal health condition. In some embodiments, the at least one other health condition corresponds to at least one of cancer, diabetes, heart failure, heart disease, chronic obstructive pulmonary disease, covid 19, and at least one other health condition.

In some embodiments, the user account associated with the user includes at least one of an electronic mail account, a pharmacy account, a health insurance account, and at least one other user account. In some embodiments, the data gathering queries include at least one questions corresponding to, at least, a mental or emotional state of the user. In some embodiments, the personalized experience interface includes one or more personalized components that include at least a daily interaction component, a relevant resource component, and a dynamic selection component. In some embodiments, the dynamic selection component includes at least one general selection and, responsive to the at least one health condition, at least one health condition specific component. In some embodiments, the personalized experience interface corresponds to a user avatar that models a biological identity of the user.

In some embodiments, the method also includes providing, at a metaverse, access to at least one three-dimensional environment feature using the user avatar. In some embodiments, the at least one three-dimensional environment feature includes at least one of a virtual healthcare provider office, a virtual healthcare provider, and at least one other three-dimensional environment feature. In some embodiments, the virtual healthcare provider includes at least one of an avatar corresponding to a healthcare provider and an avatar corresponding to an artificially intelligent healthcare provider. In some embodiments, the method also includes identifying, using a first set of user responses, a healthcare provider pool. In some embodiments, the method also includes adjusting at least some of the data gathering queries based on the first set of user responses. In some embodiments, the method also includes identifying, responsive to a second set of user responses corresponding to at least some of the adjusted data gathering queries, a subset of healthcare providers of the healthcare provider pool that match with the user.

In some embodiments, the personalized experience interface includes, at least, information corresponding to each of the subset of healthcare providers. In some embodiments, the method also includes scheduling, responsive to a selection of at least one healthcare provider of the subset of healthcare providers by the user using the personalized experience interface, at least one appointment, for the user, with the selected at least one healthcare provider. In some embodiments, the method also includes receiving feedback from the user corresponding to at least one of a component of the personalized experience interface and an appointment with the selected at least one healthcare provider. In some embodiments, the method also includes refining, responsive to the feedback, at least the data gathering queries and the personalized experience interface. In some embodiments, the method also includes identifying, based on the feedback, at least one other healthcare provider from the healthcare provider pool.

In some embodiments, the method also includes selecting, using the data structure corresponding to the user, at least one suggestive aspect of the personalized experience interface and providing, at the personalized experience interface, the at least one suggestive aspect. In some embodiments, the at least one suggestive aspect includes at least one of a color, a dynamic background, and at least one other suggestive aspect. In some embodiments, the at least one suggestive aspect is configured to provide, responsive to the user engaging with the at least one suggestive aspect, at least one of a physical response of the user and a mental response of the user. In some embodiments, the method also includes generating or refining at least one of at least one aspect of the data gathering queries and at least one aspect of the personalized experience interface, using at least one machine learning model configured to use at least some of the user responses and at least some responses provided by other users to dynamically generate or refine the at least one of at least one aspect of the data gathering queries and at least one aspect of the personalized experience interface.

In some embodiments, a system for providing an interactive digital personalized experience interface includes a processor, and a memory. The memory includes instructions that, when executed by the processor, cause the processor to: receive, responsive to a user filling at least one prescription, a prescription notification indicating information corresponding to the at least one prescription and information corresponding to the user; identify, based on the prescription notification, at least one health condition of the user; in response to the at least one health condition corresponding to at least one health condition of a plurality of predetermined health conditions, generate a message including instructions for downloading a user application; communicate, to a user account associated with the user, the message; in response to an indication that the user initiated the user application, provide, at an application setup interface, a plurality of data gathering queries; store user responses to the data gathering queries; generate, using the user responses, a data structure corresponding to the user; generate, based on the data structure corresponding to the user, a personalized experience interface; and provide, at a display of a computing device associated with the user, the personalized experience interface.

In some embodiments, the instructions further cause the processor to identify, using a first set of user responses, a healthcare provider pool. In some embodiments, the instructions further cause the processor to adjust at least some of the data gathering queries based on the first set of user responses. In some embodiments, the instructions further cause the processor to identify, responsive to a second set of user responses corresponding to at least some of the adjusted data gathering queries, a subset of healthcare providers of the healthcare provider pool that match with the user.

In some embodiments, an apparatus for providing an interactive digital personalized experience interface includes one or more processors, and a memory. The memory includes instructions that, when executed by the one or more processors, cause the one or more processors to, respectively or collectively: receive a prescription notification indicating information corresponding to at least one prescription and information corresponding to a user associated with the at least one prescription; identify, based on the prescription notification, at least one health condition of the user; in response to the at least one health condition corresponding to at least one health condition of a plurality of predetermined health conditions, generate a message including instructions for downloading a user application; communicate, to a user account associated with the user, the message; in response to an indication that the user initiated the user application, provide, at an application setup interface, a plurality of data gathering queries; generate, using the user responses, a data structure corresponding to the user; generate, based on the data structure corresponding to the user, a personalized experience interface; and provide, at a display of a computing device associated with the user, the personalized experience interface.

The present disclosure relates generally to interactive digital personalized experience interfaces which can be used with various other digital healthcare systems and methods such as U.S. patent application Ser. No. 18/204,616, titled METHODS AND SYSTEMS FOR UPDATING AND CURATING DATA; Ser. No. 18/204,716, titled RECURRING REMOTE MONITORING WITH REAL-TIME EXCHANGE TO ANALYZE HEALTH DATA AND GENERATE ACTION PLANS; Ser. No. 18/204,798, titled SURVEY AND SUGGESTION SYSTEM, Ser. No. 18/204,834, titled AUTOMATED RISK MODEL PROCESSING AND MULTIDIMENSIONAL PROVIDER MATCHING ARCHITECTURE; Ser. No. 18/205,203, titled MACHINE LEARNING MODELS FOR GENERATING EXECUTABLE SEQUENCES; and Ser. No. 18/205,249, titled SCALABLE FRAMEWORK FOR DIGITAL MESH, each of which are incorporated by reference. The predictive model generation systems can be used with the present disclosure to generate or interpret the interactive digital personalized experience interfaces.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. As used herein, the phrase at least one of A, B, and C should be construed to mean a logical (A OR B OR C), using a non-exclusive logical OR, and should not be construed to mean “at least one of A, at least one of B, and at least one of C.”

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A. The term subset does not necessarily require a proper subset. In other words, a first subset of a first set may be coextensive with (equal to) the first set.

In this application, including the definitions below, the term “module” or the term “controller” may be replaced with the term “circuit.” The term “module” may refer to, be part of, or include processor hardware (shared, dedicated, or group) that executes code and memory hardware (shared, dedicated, or group) that stores code executed by the processor hardware.

The module may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WIFI wireless networking standard) and IEEE Standard 802.3-2015 (also known as the ETHERNET wired networking standard). Examples of a WPAN are the BLUETOOTH wireless networking standard from the Bluetooth Special Interest Group and IEEE Standard 802.15.4.

The module may communicate with other modules using the interface circuit(s). Although the module may be depicted in the present disclosure as logically communicating directly with other modules, in some embodiments the module may actually communicate via a communications system. The communications system includes physical and/or virtual networking equipment such as hubs, switches, routers, and gateways. In some embodiments, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In some embodiments, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of a non-transitory computer-readable medium are nonvolatile memory devices (such as a flash memory device, an erasable programmable read-only memory device, or a mask read-only memory device), volatile memory devices (such as a static random access memory device or a dynamic random access memory device), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

Implementations of the systems, algorithms, methods, instructions, etc., described herein may be realized in hardware, software, or any combination thereof. The hardware may include, for example, computers, intellectual property (IP) cores, application-specific integrated circuits (ASICs), programmable logic arrays, optical processors, programmable logic controllers, microcode, microcontrollers, servers, microprocessors, digital signal processors, or any other suitable circuit. In the claims, the term “processor” should be understood as encompassing any of the foregoing hardware, either singly or in combination. The terms “signal” and “data” are used interchangeably.

What is claimed is:

1. A method for providing an interactive digital personalized experience interface, the method comprising:
receiving, responsive to a user filling at least one prescription, a prescription notification indicating information corresponding to the at least one prescription and information corresponding to the user;
identifying, based on the prescription notification, at least one health condition of the user;
in response to the at least one health condition corresponding to at least one health condition of a plurality of predetermined health conditions, generating a message including instructions for downloading a user application;
communicating, to a user account associated with the user, the message;
in response to an indication that the user initiated the user application, providing, at an application setup interface, a plurality of data gathering queries;
storing user responses to the data gathering queries;
generating, using the user responses, a data structure corresponding to the user;
generating, based on the data structure corresponding to the user, a personalized experience interface; and
providing, at a display of a computing device associated with the user, the personalized experience interface.

2. The method of claim 1, wherein the information corresponding to the at least one prescription includes at least prescription identification information and dosing information.

3. The method of claim 1, wherein the information corresponding to the user includes at least user statistical information and user contact information.

4. The method of claim 1, wherein the plurality of predetermined health conditions include at least one of at least one behavioral health condition, at least one musculoskeletal health condition, and at least one other health condition.

5. The method of claim 4, wherein the at least one behavioral health condition corresponds to at least one of depression, generalized anxiety, bipolar disorder, and at least one other behavioral health condition.

6. The method of claim 4, wherein the at least one musculoskeletal health condition corresponds to at least one of back pain, neck pain, joint pain, and at least one other musculoskeletal health condition.

7. The method of claim 4, wherein the at least one other health condition corresponds to at least one of cancer, diabetes, heart failure, heart disease, chronic obstructive pulmonary disease, covid 19, and at least one other health condition.

8. The method of claim 1, wherein the user account associated with the user includes at least one of an electronic mail account, a pharmacy account, a health insurance account, and at least one other user account.

9. The method of claim 1, wherein the data gathering queries include at least one questions corresponding to, at least, a mental or emotional state of the user.

10. The method of claim 1, wherein the personalized experience interface includes one or more personalized components that include at least a daily interaction component, a relevant resource component, and a dynamic selection component.

11. The method of claim 10, wherein the dynamic selection component includes at least one general selection and, responsive to the at least one health condition, at least one health condition specific component.

12. The method of claim 10, wherein the personalized experience interface corresponds to a user avatar that models a biological identity of the user.

13. The method of claim 12, further comprising providing, at a metaverse, access to at least one three-dimensional environment feature using the user avatar.

14. The method of claim 13, wherein the at least one three-dimensional environment feature includes at least one of a virtual healthcare provider office, a virtual healthcare provider, and at least one other three-dimensional environment feature.

15. The method of claim 14, wherein the virtual healthcare provider includes at least one of an avatar corresponding to a healthcare provider and an avatar corresponding to an artificially intelligent healthcare provider.

16. A system for providing an interactive digital personalized experience interface, the system comprising:
a processor; and
a memory including instructions that, when executed by the processor, cause the processor to:
receive, responsive to a user filling at least one prescription, a prescription notification indicating information corresponding to the at least one prescription and information corresponding to the user;
identify, based on the prescription notification, at least one health condition of the user;
in response to the at least one health condition corresponding to at least one health condition of a plurality of predetermined health conditions, generate a message including instructions for downloading a user application;
communicate, to a user account associated with the user, the message;
in response to an indication that the user initiated the user application, provide, at an application setup interface, a plurality of data gathering queries;
store user responses to the data gathering queries;
generate, using the user responses, a data structure corresponding to the user;
generate, based on the data structure corresponding to the user, a personalized experience interface; and
provide, at a display of a computing device associated with the user, the personalized experience interface.

17. The system of claim 16, wherein the instructions further cause the processor to identify, using a first set of user responses, a healthcare provider pool.

18. The system of claim 17, wherein the instructions further cause the processor to adjust at least some of the data gathering queries based on the first set of user responses.

19. The system of claim 18, wherein the instructions further cause the processor to identify, responsive to a second set of user responses corresponding to at least some of the adjusted data gathering queries, a subset of healthcare providers of the healthcare provider pool that match with the user.

20. An apparatus for providing an interactive digital personalized experience interface, the apparatus comprising:
one or more processors; and
a memory including instructions that, when executed by the one or more processors, cause the one or more processors to, respectively or collectively:
receive a prescription notification indicating information corresponding to at least one prescription and information corresponding to a user associated with the at least one prescription;
identify, based on the prescription notification, at least one health condition of the user;
in response to the at least one health condition corresponding to at least one health condition of a plurality of predetermined health conditions, generate a message including instructions for downloading a user application;
communicate, to a user account associated with the user, the message;
in response to an indication that the user initiated the user application, provide, at an application setup interface, a plurality of data gathering queries;
generate, using the user responses, a data structure corresponding to the user;
generate, based on the data structure corresponding to the user, a personalized experience interface; and
provide, at a display of a computing device associated with the user, the personalized experience interface.
